(19) United States
(12) Patent Application Publication
(10) Pub. No.: US 2025/0108101 A1
(43) Pub. Date: Apr. 3, 2025

(54) Non-Oncolytic Virus Infected Dead Cancer Cell Vaccine

(71) Applicant: Queen's University at Kingston, Kingston (CA)

(72) Inventors: Kyle Seaver, Woodbridge (CA); Sam Basta, Kingston (CA); Nasry Zane Bouzeineddine, Kingston (CA)

(21) Appl. No.: 18/905,433

(22) Filed: Oct. 3, 2024

Related U.S. Application Data
(60) Provisional application No. 63/542,098, filed on Oct. 3, 2023.

(51) Int. Cl. A61K 39/00; A61P 35/00; C12N 7/00

(52) U.S. Cl. CPC A61K 39/0011; A61P 35/00; C12N 7/00; A61K 2039/5152; C12N 2760/10034

(57) ABSTRACT

A composition includes at least one tumour antigen comprising dead infected tumour cells that were infected and incubated with a non-oncolytic virus prior to cell death and a pharmaceutically acceptable vehicle. The at least one tumour antigen may be a tumour associated antigen (TAA), at least one tumour specific antigen (TSA), or a combination thereof. The composition may be used as a cancer vaccine, a prophylactic cancer vaccine, or as a therapeutic cancer treatment, wherein the composition prevents, inhibits, or slows tumour development.

### RELATED APPLICATION

This application claims the benefit of the filing date of Application No. 63/542,098, filed Oct. 3, 2023, the contents of which are incorporated herein by reference in their entirety.

### FIELD

This invention relates to cancer vaccines based on dead cancer cells. More particularly, the invention relates to cancer vaccines based on dead cancer cells wherein the cancer cells were infected with a non-oncolytic virus prior to cell death.

### BACKGROUND

Cancer vaccines can be delivered in a prophylactic setting as a tumour preventative strategy [1]. There are multiple types of cancer vaccines, one of which is whole tumour cell vaccines which provide access to all potential tumour antigens that an immune response can be generated against [2, 3]. For a whole tumour cell to be delivered in vivo the cells must be dead or dying, constituting what is referred to as a dead tumour cell vaccine (DTCV). However, dead tumour cells alone may be poorly immunogenic and therefore adjuvants have been proposed to improve their efficacy [4, 5].

Use of viruses, either as adjuvants in a dead cancer cell regime, or to infect cancer cells prior to administration as a vaccine, has been proposed. Fewer studies have investigated the latter. In one study Morales et al. (2017) [13] treated mice with a vaccine prepared from tumour cells infected with infectious salmon anemia virus (ISAV), with the goal that the expressed fusogenic protein of the ISAV would be present in the dead infected tumour cells and would mediate the fusion between the dead tumour cells and the phagosome or cellular membranes of the antigen presenting cells, thereby delivering antigens to the cytoplasm enhancing cross-priming. It was observed that ISAV expressing dead tumour cells increased CD4 and CD8 cells in the spleen, but the efficacy of ISAV infection was otherwise unclear since prophylactic antitumour treatment using the dead tumour cells was beneficial independent of the expression of ISAV fusion protein. However, the potential for dead infected cancer cells to promote the presentation of tumour antigens in a prophylactic cancer vaccine is unknown.

### SUMMARY

According to one aspect of the invention there is provided a composition, comprising: at least one tumour antigen comprising dead infected tumour cells that were infected and incubated with a non-oncolytic virus prior to cell death; and a pharmaceutically acceptable vehicle.

In various embodiments the at least one tumour antigen comprises at least one tumour associated antigen (TAA), at least one tumour specific antigen (TSA), or a combination of at least one TAA and at least one TSA.

In various embodiments the composition is a cancer vaccine. The composition may be a prophylactic cancer vaccine, a therapeutic cancer treatment, or a combination thereof.

In one embodiment the non-oncolytic virus comprises lymphocytic choriomeningitis virus (LCMV).

In one embodiment the dead infected tumour cells comprise γ-irradiated tumour cells.

In one embodiment the dead infected tumour cells comprise lysis and UV treated tumour cells.

In one embodiment the dead infected tumour cells comprise B16 tumour cells.

In one embodiment the dead infected tumour cells comprise B16-OVA tumour cells.

In one embodiment the dead infected tumour cells comprise tumour cells that were incubated with the non-oncolytic virus for at least 24 hours prior to cell death.

According to another aspect of the invention there is provided a method for preventing, inhibiting, or slowing tumour development, comprising: providing a composition comprising at least one tumour antigen comprising dead infected tumour cells that were infected and incubated with a non-oncolytic virus prior to cell death, and a pharmaceutically acceptable vehicle; administering an effective amount of the composition to a subject; wherein the composition prevents, inhibits, or slows tumour development in the subject.

In various embodiments the at least one tumour antigen comprises at least one tumour associated antigen (TAA), at least one tumour specific antigen (TSA), or a combination of at least one TAA and at least one TSA.

The method may comprise administering the composition to the subject prophylactically.

The method may comprise administering the composition to the subject therapeutically.

In one embodiment the non-oncolytic virus comprises lymphocytic choriomeningitis virus (LCMV).

In one embodiment the dead infected tumour cells comprise γ-irradiated tumour cells.

In one embodiment the dead infected tumour cells comprise lysis and UV treated tumour cells.

In one embodiment the dead infected tumour cells comprise B16 tumour cells.

In one embodiment the dead infected tumour cells comprise B16-OVA tumour cells.

According to another aspect of the invention there is provided a method for enhancing efficacy of a dead tumour cell vaccine (DTCV), comprising: infecting and incubating tumour cells with a non-oncolytic virus; exposing the infected and incubated tumour cells to at least one treatment that causes cell death without substantially reducing activity of the dead tumour cells as a tumour antigen; wherein the resulting dead infected tumour cells have enhanced efficacy as a DTCV relative to dead tumour cells that were not infected and incubated with a non-oncolytic virus.

In one embodiment the at least one treatment that causes cell death comprises γ-irradiation or lysis and UV irradiation.

In one embodiment the non-oncolytic virus comprises lymphocytic choriomeningitis virus (LCMV).

In one embodiment the tumour cells comprise B16 tumour cells comprising at least one TAA, at least one TSA, or a combination thereof.

In one embodiment the tumour cells comprise B16-OVA tumour cells or EL4-OVA tumour cells comprising at least one TAA, at least one TSA, or a combination thereof.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A and 1B are fluorescence microscopy images of B16 melanoma cells and murine macrophage cell line (BMA3.1A7) cells infected with LCMV-ARM-GFP, respectively.

FIGS. 1C and 1D are representative flow cytometry histograms of B16 cells infected with LCMV-ARM-GFP and LCMV-ARM, respectively, wherein infection by LCMV-ARM was detected by nuclear protein (NP) staining; percentage of positive cells is shown in the top right of each histogram. Data is from three independent experiments.

FIGS. 1E and 1F are representative histograms of BMA cells infected with LCMV-ARM-GFP and LCMV-NP, respectively, wherein infection by LCMV-ARM was detected by nuclear protein (NP) staining; percentage of positive cells is shown in the top right of each histogram. Data is from three independent experiments.

FIG. 2 is a plot showing the impact of LCMV infection on cell viability in B16 cells compared to uninfected control cells at varying time points after infection; data are ±SD of four independent experiments.

FIG. 3A is a representative histogram of B16 cells stained for LCMV-NP, confirming that greater than 50% of the B16 cells were infected prior to γ-irradiation (60 Gys) exposure.

FIG. 3B is a schematic representation of a vaccination preparation and protocol scheme, according to one embodiment.

FIG. 3C is a Kaplan-Meier survival curve for mice that received vaccinations according to one embodiment, and controls that received PBS, where **p<0.005 and ns denotes not significant.

FIG. 3D is plot of tumour growth in mice that received vaccinations according to one embodiment, and controls that received PBS, followed by live B16 challenge.

FIGS. 4A-4L show results of splenocyte analyses for mice that received vaccinations according to one embodiment, and controls that received PBS; data is presented as ±SD with three mice per group; *p<0.05, **p<0.01, ***p<0.001, ****p<0.0001; wherein:

FIGS. 4A and 4B are plots of percentage of CD3+ cells that expressed CD8 or CD4, respectively;

FIGS. 4C and 4D are plots of mean fluorescence intensity of CD62L of CD3+CD8+ or CD3+CD4+ cells, respectively;

FIGS. 4E and 4F are plots of mean fluorescence intensity of CD69 of CD3+CD8+ or CD3+CD4+ cells, respectively;

FIG. 4G is a plot of percentage of CD3− cells that express NK1.1;

FIGS. 4H and 4I are plots of mean fluorescence intensity of CD62L and CD69 of CD3−NK1.1+ cells, respectively; and

FIGS. 4J-4L are plots of mean fluorescence intensity of CD80, CD86, and PD-L1, respectively, of CD11c+MHC-II+ cells.

### DETAILED DESCRIPTION OF EMBODIMENTS

Embodiments described herein provide a cancer vaccine based on dead cancer cells, wherein the cancer cells were infected with a non-oncolytic virus prior to cell death and the dead infected cancer cells are effective at activating antigen-presenting cells (APCs) that may be used prophylactically in a cancer vaccine and/or therapeutically in a cancer treatment.

As used herein, the term “non-oncolytic” in respect of a virus means a virus that does not cause lysis in a tumour cell and may or may not cause lysis in a non-tumour cell.

As used herein, the term “tumour associated antigen” or “TAA” refers to an antigenic feature on a tumour cell or a normal cell, examples of which may include, but are not limited to, a molecule such as a protein, embryonic protein, glycoprotein, a squamous cell antigen, etc., against which an immune response is induced.

As used herein, the term “tumour specific antigen” or “TSA” refers to a protein or other molecule that is found only on cancer cells and not on normal cells. Tumour specific antigens can help the body make an immune response against cancer cells. They may be used as targets for targeted therapy or for immunotherapy to help boost the body's immune system to kill more cancer cells. Tumour specific antigens may also be used in tests to diagnose some types of cancer.

As used herein, the term “tumour antigen” refers to at least one TAA, or at least one TSA, or a combination of at least one TAA and at least one TSA.

As used herein, the term “dead tumour cell” refers to a tumour cell that has been subjected to a treatment that renders the cell non-viable (e.g., incapable of replicating) and preserves tumour cell antigenic features that contribute to efficacy of a vaccine as described herein. Such tumour cell antigenic features may include, for example, surface characteristics such as one or more cell surface proteins and/or other proteins (i.e., TAAs) against which an immune response is induced in a subject having received a dead cancer cell vaccine according to an embodiment. For example, dead tumour cells used in embodiments may be prepared by subjecting tumour cells to one or more treatments such as, but not limited to, irradiation (e.g., γ-irradiation, ultra-violet (UV) irradiation, or irradiation using other wavelengths, such as visible light) or a treatment that induces apoptosis such as plasma irradiation, or lysis (e.g., freeze/thaw cycles). In one embodiment, dead tumour cells may be prepared by a combination of lysis and UV irradiation (“LyUV”). The dead tumour cells, which may also be referred to as antigen donor cells, may be suitably purified for inclusion in a cancer vaccine composition to be administered to a subject.

The use of oncolytic viruses (OVs) has been proposed in prior work, for example, to induce death in cancer cells wherein the resulting dead cells may be immunogenic, and additionally wherein the oncolytic virus can act as an adjuvant by stimulating immune cells (e.g., through Toll-like receptor (TLR) activation [6, 7]). The cell death may activate an immune response owing to the release of danger-associated molecular patterns (DAMPs). In addition to killing cancer cells, OVs can also directly activate an innate immune response as pattern recognition receptors (PRRs) recognize the pathogen-associated molecules patterns (PAMPs) associated with the OV. However, OVs can be limited as the cancer cells must express the receptor to permit viral entry, and the number of OVs is far less than non-OVs. It is recognized herein that whether OVs and oncolysis are beneficial in a prophylactic setting, where a tumour is absent, is unclear. In contrast, by using a non-oncolytic virus, embodiments described herein are effective in a prophylactic cancer vaccine.

Lymphocytic choriomeningitis virus (LCMV) is an example of a non-oncolytic virus that may be used according to embodiments described herein wherein the virus improves the efficacy of a DTCV. In one embodiment, LCMV improves the efficacy of a DTCV delivered in a prophylactic setting against B16 melanoma. Furthermore, according to some embodiments, LCMV-infected B16 cells may be more effective at activating immune cells in vivo when compared to an uninfected DTCV. Embodiments demonstrate the use of non-oncolytic viruses in combination with DTCVs to establish an anti-tumour immune response in a prophylactic setting that can protect against a tumour challenge. Embodiments also demonstrate the use of non-OVs as an alternative method to enhance whole tumour cell vaccine efficacy in immunotherapy.

As used herein, the term “administration” of a cancer vaccine composition to a subject includes any route or routes of introducing or delivering to a subject the composition to perform its intended function. Administration can be carried out by any suitable route, including orally, intranasally, intraocularly, parenterally (intravenously, intramuscularly, intrathecally, epidurally, intracranially, intraperitoneally, or subcutaneously), or topically, or by a combination thereof. Parenteral administration of the composition is generally characterized by intra-tumour injection. Injectables can be prepared in conventional forms, either as liquid solutions or suspensions, solid forms suitable for solution of suspension in liquid prior to injection, or as emulsions.

The exact amount of a composition required will vary from subject to subject, depending on the species, age, weight, and general condition of the subject, the stage and location of the cancer being treated, the mode of administration, and the like. Thus, it is not possible to specify an exact amount for every composition. However, an appropriate amount can be determined by one of ordinary skill in the art using only routine experimentation and known methods, such as dose titration, given the teachings herein.

As used herein, the term “effective amount” or “pharmaceutically effective amount” or “therapeutically effective amount” or “prophylactically effective amount” of a composition is a quantity sufficient to achieve or maintain a desired therapeutic and/or prophylactic effect, e.g., an amount which results in the prevention of, or a decrease in, the cancer being treated, e.g., of a tumour. An effective amount can be determined by one of ordinary skill in the art using only routine experimentation and known methods, such as dose titration, given the teachings herein.

As described herein, the compositions may be administered in vivo in a pharmaceutically acceptable carrier. The term “pharmaceutically acceptable” refers to a substance that is not biologically or otherwise undesirable, i.e., the substance is physiologically compatible and may be administered to a subject without causing any undesirable biological effects or interacting in a deleterious manner with any of the other components of the pharmaceutical composition in which it is contained. The carrier would be selected to minimize any degradation of the active ingredient(s) and to minimize any adverse side effects in the subject, as would be well known to one of ordinary skill in the art.

As used herein, the term “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers and their formulations are described in Remington: The Science and Practice of Pharmacy (19th ed.) ed. A. R. Gennaro, Mack Publishing Company, Easton, Pa. 1995. For example, an appropriate amount of a pharmaceutically acceptable salt may be used in the formulation to render the formulation isotonic. Examples of the pharmaceutically acceptable carriers include, but are not limited to, saline, Ringer's solution, and dextrose solution. The pH of the solution is preferably from about 5 to about 8, and more preferably from about 7 to about 7.5. Further carriers may include sustained release preparations such as semipermeable matrices of solid hydrophobic polymers containing the active ingredient(s), which matrices are in the form of shaped articles, e.g., films, liposomes, or microparticles. It will be apparent to those of ordinary skill in the art that certain carriers may be more preferable depending upon, for instance, the route of administration and concentration of composition being administered. The invention will be further described by way of the below examples wherein it is demonstrated that embodiments comprising γ-irradiated dead tumour cells

Embodiments will be further described by way of the below examples. Specific features described in the examples, such as dosage and administration schedule, may be varied, and the approach may be adapted for other types of cancers and other subjects, as would be apparent to those of ordinary skill in the art. Accordingly, the examples are not to be construed as limiting the invention in any way.

### EXAMPLE 1

### 1. Methods

### Mice and Cell Lines

Six to eight-week-old C57BL/6 (H-2^{b}) mice (male and female) were used for tumour engraftment studies. Mice were purchased from JAX® Laboratories (Bar Harbour, USA). All animals were housed under specific pathogen-free conditions. According to the Canadian Council of Animal Use, Experiments were carried out and approved by Queen's University Animal Care Services.

The murine macrophage cell line BMA3.1A7 (BMA) was used, which originated from bone marrow cells isolated from adult female C57BL/6 mice and were immortalized by overexpressing the raf and myc oncogenes (these cells were a gift from Dr. Ken L. Rock, University of Massachusetts Medical School, Worcester, USA). The BMA cells were cultured in RPMI (Gibco, Fisher Scientific, Canada) media supplemented with 5% FBS (Gibco, Fisher Scientific, Canada). For tumour engraftment studies, the B16F10 (H-2k^{b}) melanoma cell lines were used and maintained in DMEM media (Gibco, Fisher Scientific, Canada) supplemented with 5% FBS. All cells were maintained at 37° C. and 5% CO₂. The BMA cells were used as a positive control to determine whether the virus could replicate in the infected cells by detecting GFP signal (see below). The approach was extended to the B16 melanoma cell line to determine if cancer cells could be infected.

### Virus Preparation

Both LCMV Armstrong (LCMV-ARM) and the recombinant LCMV-ARM strain expressing green fluorescent protein (LCMV-ARM-GFP) were propagated using baby hamster kidney (BHK) fibroblast cell lines which were obtained from F. Lehmann-Grube (Germany). The LCMV-ARM-GFP was originally prepared in Dr. De La Torre's lab at the Scripps Research Institute (La Jolla, USA) [8], and kindly provided by Dr. Watt from the University of Toronto (Ontario, Canada). Virus titration was carried out as previously described [9]. Following this viral titration protocol, the resulting virus titration was determined to be 2.0×10⁶.

### Detection of LCMV-NP and LCMV-GFP Fluorescence Microscopy or by Flow Cytometry

To determine LCMV-GFP infection, fluorescence microscopy or flow cytometry was utilized. For fluorescence microscopy, B16 or BMA cells were seeded into a 12-well plate (Corning, USA) at a density of 2.0×10⁶ cells/well. Cells were infected at a multiplicity of infection (MOI) of 1 and incubated for 96 hours, after which cells were visualized by fluorescence microscopy (Lecia DM IRE2, Germany) at 20× magnification. For fluorescence images, the UV extraction filter N2.1S was used with the Lecia DFC340-cooled monochrome digital camera. For LCMV-GFP detection by flow cytometry, cells were harvested following the indicated incubation time. Cells were washed in 1×PBS (Gibco, Fisher Scientific, Canada) and were analyzed by the CytoFLEX flow cytometer (Beckman Coulter, USA) using the 488 nm excitation laser and the 525 nm emission detection filter. For the detection of LCMV-NP, cells were harvested after the indicated incubation time and washed in 1×PBS, after which cells were fixed using 4% paraformaldehyde (PFA) for 15 min at room temperature. The fixed cells were then permeabilized with 0.1% saponin in PBS for 20 min at 4° C. Cells were washed in PBS and stained with anti-LCMV-NP antibody (clone VL4) supernatants (a gift from Dr. M. Groettrup, Germany) for 1 h at room temperature as previously outlined [10]. After cells were washed with 0.1% saponin in PBS to remove excess antibody and stained with FITC-conjugated goat anti-rat IgG antibody (Invitrogen, USA) for 30 min and room temperature in 0.1% saponin. Samples were acquired on the CytoFLEX and analyzed using the FlowJo software (BD, USA).

### B16 Infection for Cell Viability Analysis

B16 cells were seeded into a 12-well plate at a density of 2.0×10⁵ cells/well. Cells were then infected with LCMV-ARM or LCMV-ARM-GFP for the indicated amount of time at an MOI of 1. To assess cell viability, cells were incubated with 7-AAD as per the manufacturer's recommendation (Biolegend, USA). Briefly, cells were seeded into 0.5 mL of flow running (1×PBS, 0.1% sodium azide) and 5 μL of 7-AAD was added and cell were incubated for 10 minutes (in the dark) before acquisition by flow cytometry. Dead cells were detected as being 7-AAD positive using the 488 nm excitation laser and the 690 nm emission filter on the CytoFLEX flow cytometer, samples were analyzed using FlowJo software (BD, USA).

### Preparation of an LCMV-Infected B16 DTCV

B16 cells were isolated and either left uninfected or infected with LCMV-ARM at an MOI of 0.2 and incubated for 48 h, after which the percent of infected cells was confirmed prior to proceeding. After confirmation of infection (greater than 50% of the cells) cell were washed five times in 1×PBS and 5.0×10⁶ cells were resuspended in 200 μL of 1×PBS.

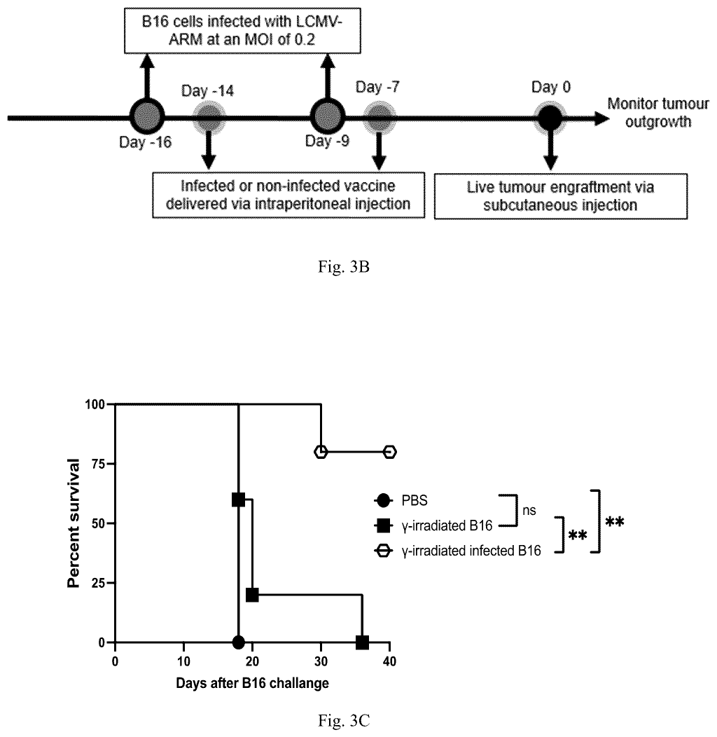

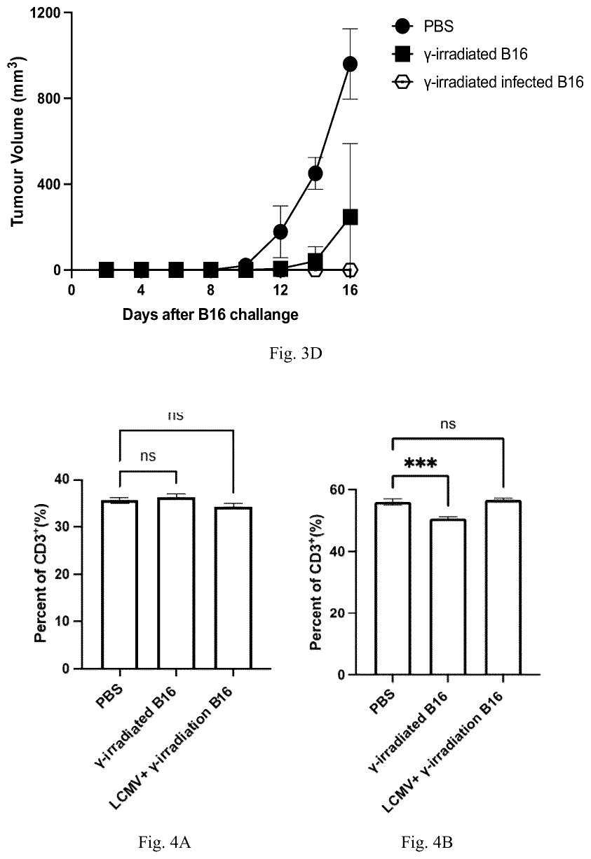

Various methods may be used to induce cell death, but the specific method used can impact an ensuing anti-tumour immune response [11]. Exposure to γ-irradiation is a common method used to generate DTCV, as the cells remain intact and can be modified to result in the secretion of desired cytokines such as GM-CSF [12], and was selected as the method to be used for these experiments. Cells were exposed to 60 Gys of γ-irradiation using a cesium irradiator as the source (Cs¹³⁷ Gamma Cell 20 Irradiator, Queen's University). A total of 5.0×10⁶ infected or uninfected B16 cells were injected intraperitoneally (i.p) on days −14 and −7. On day 0, the mice were injected with 1.0×10⁶ live B16 cells. Tumour outgrowth was measured every second day using callipers and tumour volume was determined by V=(L×W²)/2 until tumour volume reached 2000 mm³. FIG. 3B is a schematic representation of the vaccination protocol used according to this embodiment.

### Flow Cytometry Analysis

For in vivo analysis of cell activation, LCMV-ARM infected or uninfected B16 cells were exposed to γ-irradiation and then injected i.p. Mice were euthanized and spleens and peritoneal fluid were isolated from vaccinated mice 24 h after vaccination. Isolated cells were made into a single cell suspension and washed twice in flow staining buffer (1×PBS, 0.1% sodium azide, 1% BSA) and then stained with the following antibodies for 20 min at 4° C. The following antibodies were used; anti-CD8 (clone:53-6.7), anti-CD4 (clone:GK1.5), anti-CD3 (clone:17A2), anti-CD62L (clone:MEL-14), anti-CD69 (clone:H1.2F3), anti-NK1.1 (clone:PK136). All antibodies were purchased from BioLegend. After staining, cells were washed in flow running buffer (1×PBS, 0.1% sodium azide) analyzed by flow cytometry using the CytoFLEX and analyzed using FlowJo software.

### Statistical Analysis

Statistical significance was determined using GraphPad Prism. Comparison between two groups was done using Student's t-test. One-way ANOVA was used when comparing differences between more than two groups. For Kaplan-Meier survival curves the Log-rank (Mantel-Cox) test was used. All values are reported as mean±SD. A p-value≤0.05 was considered significant.

### 2. Results

### B16 Cells Are Readily Infected With LCMV

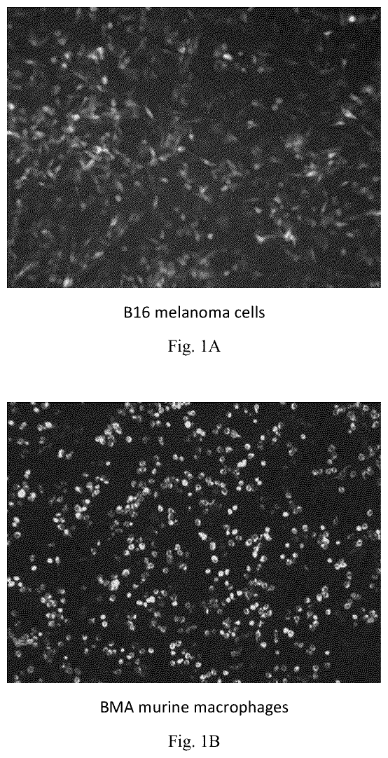

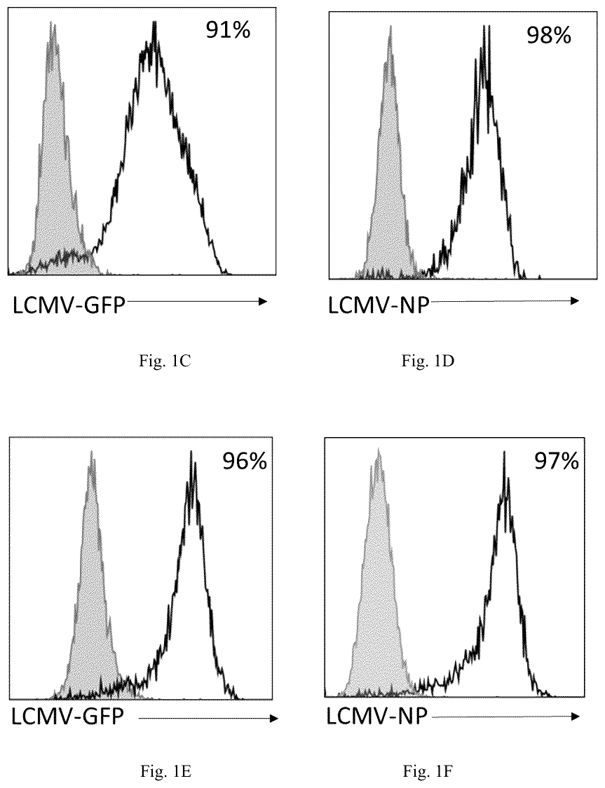

Before proceeding with in vivo models, experiments confirmed that B16 cells could be infected with LCMV. Using the LCMV-ARM model labelled with GFP, B16 cells were infected at an MOI of 1, and visualized at 20× magnification 96 hours later for infection. At early time points infection was not observed, indicating that B16 cells could support LCMV replication. Using fluorescent microscopy it was determined that the majority of B16 cells were infected 96-hours post-infection (FIG. 1A). The percentage of infected cells was quantified by flow cytometry and compared to LCMV-ARM in the absence of GFP to determine if the presence of GFP impacted the rate of infection. It was found that both LCMV-ARM with and without GFP were able to infect B16 cells at an MOI of 1 after 96 hours as seen by the histograms with greater than 90% of the cells infected (FIGS. 1C and 1D). Using non-GFP labelled virus allowed for the assessment of whether GFP impacted viral infection or replication in B16 cells. To test for LCMV infection by non-GFP virus, nuclear protein (NP) staining was used. Similar results were obtained in the BMA cell line, at the same MOI and time point, again with most cells being visually infected (FIG. 1B), and greater than 90% of the cells being infected following flow cytometry analysis FIGS. 1E and 1F). Taken together these results indicate that LCMV-ARM can readily infect B16 cells comparable to that of the BMA cell line and that GFP does not impact LCMV-ARM infection.

### LCMV Infection Does Not Impact B16 Cell Viability

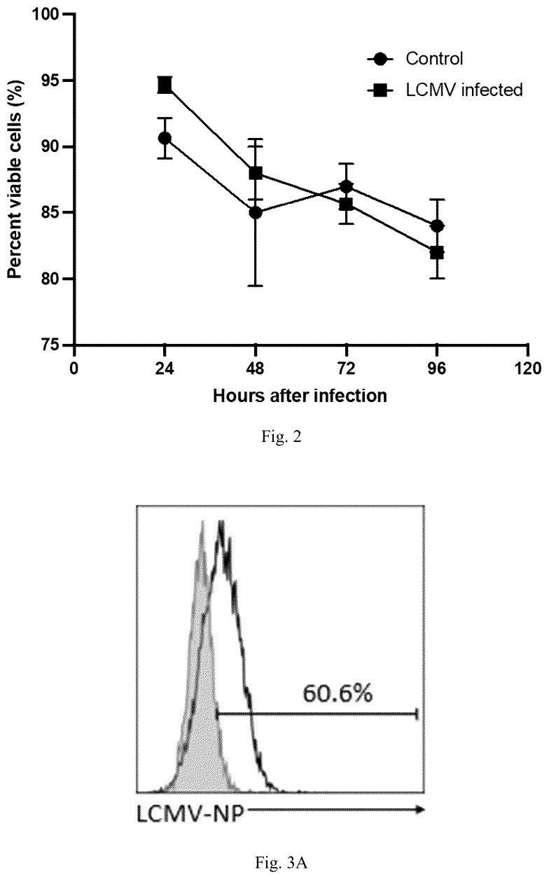

With LCMV being classified as a non-lytic virus to tumour cells (i.e., non-oncolytic), experiments investigated whether LCMV impacts B16 cell viability after various times post-infection (24-96 h). It was determined that LCMV infection did not decrease cell viability at any of the time points tested (FIG. 2). Thus, results demonstrate the potential for B16 cells to be infected with LCMV, and that LCMV does not impact the viability of these cells. Therefore, LCMV can be used as a non-oncolytic virus in B16 cells and is expected to be efficacious as an adjuvant for enhancing the efficacy of a DTCV.

### An LCMV Infected B16 DTCV Provides Improved Protection Against Tumour Challenge

With B16 cells being readily infected with LCMV, experiments investigated whether LCMV-infected cells exposed to γ-irradiation could be used as a DTCV to protect against a tumour challenge. B16 cells (5.0×10⁶ cells) were infected at an MOI of 0.2 and incubated for 48-hours, where LCMV infection of greater than 50% was confirmed by NP staining and flow cytometry (FIG. 3A). After confirmation of infection, infected B16 cells were exposed to 60 Gys of γ-irradiation and injected into mice following the vaccination schedule outlined in FIG. 3B. It was determined that LCMV infected cells significantly enhanced the survival of mice, with 80% of mice remaining tumour free after 40 days (FIG. 3C). Furthermore, both the uninfected and LCMV-infected DTCV could slow tumour growth compared to PBS vaccinated control mice, with the LCMV-infected DTCV providing the greatest reduction in tumour growth (FIG. 3D).

### LCMV Infected Cells Alter T Cells, NK Cells and Antigen-Presenting Cells in the Spleen

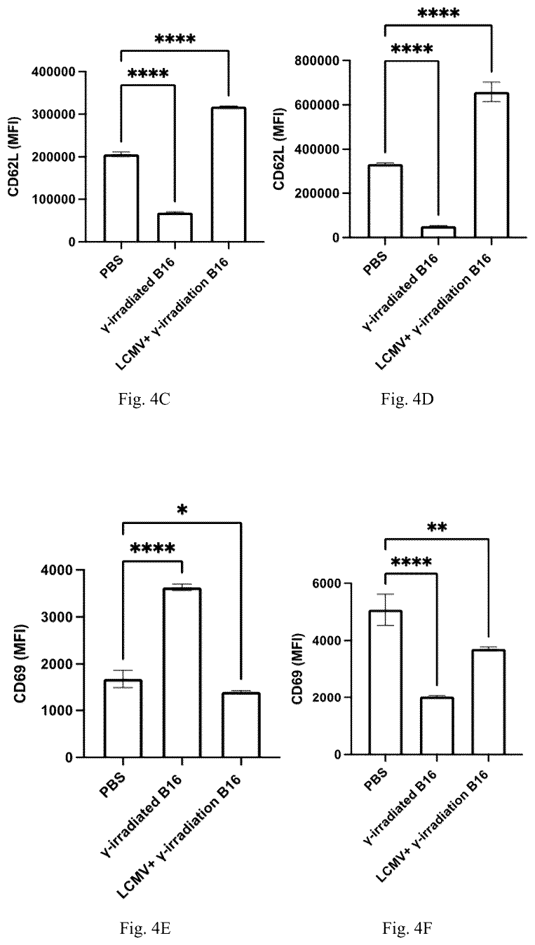

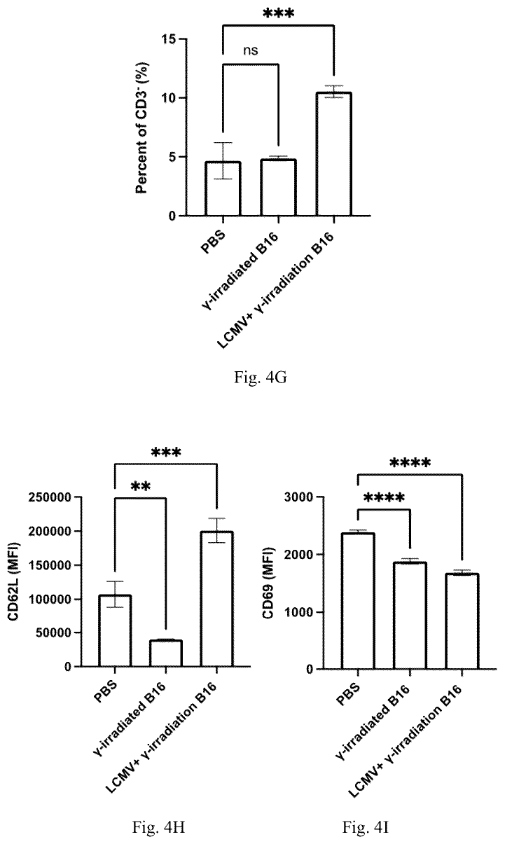

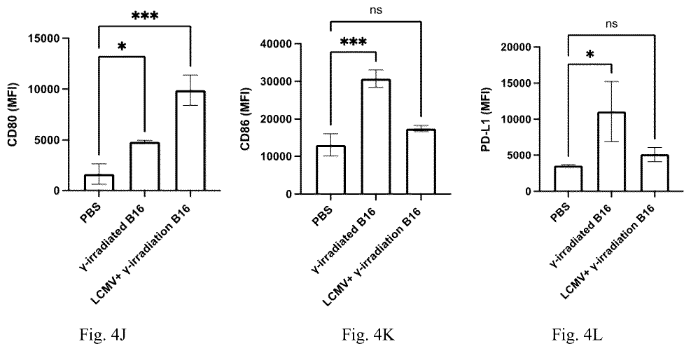

After evaluating protection, experiments investigated how LCMV-infected γ-irradiated cells impacted immune cell activation. Mice were vaccinated and 24 h after vaccination spleens were isolated to determine changes in the percentage of cells and evaluate the activation state of these cells. It was found that γ-irradiated B16 cells infected with LCMV had no change in CD8⁺ or CD4⁺ T cell percentages compared to control (FIGS. 4A and 4B). LCMV infection did result in a significant increase of CD62L (FIGS. 4C and 4D), but there was a significant decrease in CD69 expression compared to unvaccinated control mice in both CD8⁺ and CD4⁺ T cells (FIGS. 4E and 4F). The LCMV infected DTCV had a significant increase in NK cells (FIG. 4G), which had a higher expression of CD62L (FIG. 4H), but lower expression of CD69 (FIG. 4I). Finally, activation state of APCs was evaluated, specifically focusing on a more dendritic cell-like cell type classified as being CD11c⁺ and MHC-II⁺. It was found that an LCMV-infected B16 DTCV resulted in an increase of CD80 on dendritic cells (DCs) in the spleen, but there was no significant change in CD86 or PD-L1 compared to PBS control mice (FIGS. 4J, 4K, 4L). Together these results indicate that an LCMV-infected DTCV based on γ-irradiated B16 cells can alter the activation of T cells, NK cells, and DCs in the spleen as early as 24 h after vaccination.

### 3. Conclusion

The results clearly indicate that an LCMV infected DTCV can be used to protect against tumour challenges, with 80% of mice remaining tumour free 40 days after live tumour challenge. This resulted in a significant improvement in the protection provided by LCMV-infected DTCV, compared to the DTCV alone. Mice that received the infected DTCV, or the DTCV alone demonstrated slowed tumour growth compared to PBS (unvaccinated) control mice.

Overall, the results support the use of non-oncolytic viruses in an alternative approach to a DTCV. In the example of LCMV-infected DTCV prepared with γ-irradiated B16 melanoma cells, results included the generation of an anti-tumour immune response that protected against a live tumour challenge in which early changes in the activation of T cells, NK cells, and DCs were observed. The approach is applicable to other tumour models and use of other non-OVs in the preparation of DTCVs.

### EXAMPLE 2

This example provides a melanoma tumour vaccine against a lymphoma model.

A whole tumour cell vaccine was prepared by infecting B16-OVA cells with LCMV-ARM or LCMV-ARM-GFP. Fluorescence microscopy and flow cytometry were used to determine the percentage of cells infected. When the B16-OVA cells were observed to be at least about 80% infected, they were subjected to LyUV treatment. LyUV treatment involved a single round of freeze/thaw followed by immediate exposure to 10,000 mj/cm³ UV for 10 minutes. It was determined that B16-OVA cells are permissible to LCMV infection. In addition to B16-OVA, an alternative cancer cell line was tested to determine if other cancer types are permissible to LCMV infection. For this, EL4-OVA cells, a lymphoma model, and B16-OVA cells were used and it was determined that LCMV-GFP did not readily infect the EL4-OVA cells. Confirmation was obtained through flow cytometry and fluorescence microscopy, by infecting B16-OVA and EL4-OVA cells with LCMV-ARM-GFP at an MOI of 3 for 24 h and an MOI of 0.5 for 48 h. In both experimental conditions, no discernable GFP signal was detected in EL4-OVA cells.

Using LCMV as an adjuvant is feasible because the single stranded RNA can act as a TLR7 agonist, promoting antigen presenting cell activation. By using LCMV, these experiments aimed to determine if an infected DTCV consisting of B16-OVA cells can be used in a prophylactic setting to protect against a tumour challenge of a different tumour cell type expressing a similar antigen, in this case, EL4-OVA.

Four C57BL/6 mice received two doses of the vaccine, which consisted of 5.0×10⁶ LCMV-GFP-infected B16-OVA cells that were subsequently exposed to LyUV treatment. The vaccine was delivered twice two weeks apart. Two mice were used as controls receiving PBS rather than an infected DTCV. After vaccination, all mice were challenged with 1×10⁶ B16-OVA cells subcutaneously into the right hind flank, and tumour growth was monitored. Following tumour challenge, two vaccinated mice presented a tumour roughly 20 days after the unvaccinated mice presented a tumour, 40 days after engraftment. The vaccinated mice also had slower tumour growth compared to the unvaccinated mice. The remaining two vaccinated mice did not show palpable tumours 60 days after tumour engraftment. This indicated that an infected B16-OVA DTCV could protect against B16-OVA tumour challenge, with 50% of the mice remaining tumour free 60 post tumour engraftment.

In another trial, the infected B16-OVA DTCV was tested to determine if it could protect against a different tumour cell line, the EL4-OVA lymphoma cell line. In this trial, four C57BL/6 mice received the vaccine in two doses of 5.0×10⁶ cells per vaccination, one dose per week, and two mice were used as a control and received only PBS. After vaccination, the mice were challenged with 5.0×10⁶ EL4-OVA cells subcutaneously into the right hind flank. Here, one vaccinated mouse presented a tumour six days after the unvaccinated mice presented a palpable tumour, and two vaccinated mice presented a tumour about 20 days after the unvaccinated mice. Furthermore, one vaccinated mouse demonstrated complete protection with no detectable tumour 60 days after the challenge. This indicates that the infected B16-OVA DTCV may reduce the onset of tumour growth against EL4-OVA.

It is suggested herein that the vaccine being prepared with approximately 80% of the tumour cells infected with LCMV may cause some immune response competition between the tumour antigens and the viral antigens, possibly shifting the attention of the immune response towards the viral antigens. In a previous experiment where the vaccine was prepared with 50% infected cells against the same tumour model (i.e., B16-OVA vaccine against B16-OVA challenge), there was better protection than shown in the results presented here. Nevertheless, the results indicate that the vaccine may provide protection and/or slow onset of tumour growth, suggesting that the OVA antigen may increase survivability.

The contents of all cited publications are incorporated herein by reference in their entirety.

### EQUIVALENTS

It will be appreciated that modifications may be made to the embodiments described herein without departing from the scope of the invention. Accordingly, the invention should not be limited by the specific embodiments set forth but should be given the broadest interpretation consistent with the teachings of the description as a whole.

### REFERENCES

1. Sobol, R.E., The rationale for prophylactic cancer vaccines and need for a paradigm shift. Cancer Gene Ther, 2006. 13(8):725-31.
2. Keenan, B.P. and E.M. Jaffee, Whole cell vaccines—past progress and future strategies. Semin Oncol, 2012. 39(3):276-86.
3. Chiang, C.L., F. Benencia, and G. Coukos, Whole tumor antigen vaccines. Semin Immunol, 2010. 22(3):132-43.
4. Bowen, W.S., et al., Current challenges for cancer vaccine adjuvant development. Expert Rev Vaccines, 2018. 17(3):207-215.
5. Cuzzubbo, S., et al., Cancer Vaccines: Adjuvant Potency, Importance of Age, Lifestyle, and Treatments. Front Immunol, 2020. 11:615240.
6. Guilliams, M., et al., Alveolar macrophages develop from fetal monocytes that differentiate into long-lived cells in the first week of life via GM-CSF. Journal of Experimental Medicine, 2013. 210(10):1977-1992.
7. Yamano, T., et al., Whole cell vaccination using immunogenic cell death by an oncolytic adenovirus is effective against a colorectal cancer model. Mol Ther Oncolytics, 2016. 3:16031.
8. Emonet, S.F., et al., Generation of recombinant lymphocytic choriomeningitis viruses with trisegmented genomes stably expressing two additional genes of interest. Proc Natl Acad Sci USA, 2009. 106(9):3473-8.
9. Korns Johnson, D. and D. Homann, Accelerated and improved quantification of lymphocytic choriomeningitis virus (LCMV) titers by flow cytometry. PLoS One, 2012. 7(5):e37337.
10. Mulder, R., et al., M(IL-4) Tissue Macrophages Support Efficient Interferon-Gamma Production in Antigen-Specific CD8(+) T Cells with Reduced Proliferative Capacity. Front Immunol, 2017. 8:1629.
11. Scheffer, S.R., et al., Apoptotic, but not necrotic, tumor cell vaccines induce a potent immune response in vivo. Int J Cancer, 2003. 103(2):205-11.
12. Dranoff, G., et al., Vaccination with irradiated tumor cells engineered to secrete murine granulocyte-macrophage colony-stimulating factor stimulates potent, specific, and long-lasting anti-tumor immunity. Proc Natl Acad Sci USA, 1993. 90(8):3539-43.
13. Morales, J., et al., Dead Tumor Cells Expressing Infectious Salmon Anemia Virus Fusogenic Protein Favor Antigen Cross-Priming In Vitro. Front Immunol, 2017. 8:1-11.

1. A composition, comprising:
at least one tumour antigen comprising dead infected tumour cells that were infected and incubated with a non-oncolytic virus prior to cell death; and
a pharmaceutically acceptable vehicle.

2. The composition of claim 1, wherein the at least one tumour antigen comprises at least one tumour associated antigen (TAA), at least one tumour specific antigen (TSA), or a combination of at least one TAA and at least one TSA.

3. The composition of claim 1, wherein the composition is a cancer vaccine.

4. The composition of claim 1, wherein the composition is a prophylactic cancer vaccine.

5. The composition of claim 1, wherein the composition is a therapeutic cancer treatment.

6. The composition of claim 3, wherein the cancer vaccine prevents, inhibits, or slows tumour development.

7. The composition of claim 1, wherein the non-oncolytic virus comprises lymphocytic choriomeningitis virus (LCMV).

8. The composition of claim 1, wherein the dead infected tumour cells comprise γ-irradiated tumour cells.

9. The composition of claim 1, wherein the dead infected tumour cells comprise lysis and UV treated tumour cells.

10. The composition of claim 1, wherein the dead infected tumour cells comprise B16 tumour cells.

11. The composition of claim 1, wherein the dead infected tumour cells comprise B16-OVA tumour cells.

12. The composition of claim 1, wherein the dead infected tumour cells comprise tumour cells that were incubated with the non-oncolytic virus for at least 24 hours prior to cell death.

13. A method for preventing, inhibiting, or slowing tumour development, comprising:
providing a composition comprising at least one tumour antigen comprising dead infected tumour cells that were infected and incubated with a non-oncolytic virus prior to cell death, and a pharmaceutically acceptable vehicle;
administering an effective amount of the composition to a subject;
wherein the composition prevents, inhibits, or slows tumour development in the subject.

14. The method of claim 13, wherein the at least one tumour antigen comprises at least one tumour associated antigen (TAA), at least one tumour specific antigen (TSA), or a combination of at least one TAA and at least one TSA.

15. The method of claim 13, comprising administering the composition to the subject prophylactically.

16. The method of claim 13, comprising administering the composition to the subject therapeutically.

17. The method of claim 13, wherein the non-oncolytic virus comprises lymphocytic choriomeningitis virus (LCMV).

18. The method of claim 13, wherein the dead infected tumour cells comprise γ-irradiated tumour cells.

19. The method of claim 13, wherein the dead infected tumour cells comprise lysis and UV treated tumour cells.

20. The method of claim 13, wherein the dead infected tumour cells comprise B16 tumour cells.

21. The method of claim 13, wherein the dead infected tumour cells comprise B16-OVA tumour cells.

22. A method for enhancing efficacy of a dead tumour cell vaccine (DTCV), comprising:
infecting and incubating tumour cells with a non-oncolytic virus;
exposing the infected and incubated tumour cells to at least one treatment that causes cell death without substantially reducing activity of the dead tumour cells as a tumour antigen;
wherein the resulting dead infected tumour cells have enhanced efficacy as a DTCV relative to dead tumour cells that were not infected and incubated with a non-oncolytic virus.

23. The method of claim 22, wherein the at least one treatment that causes cell death comprises γ-irradiation or lysis and UV irradiation.

24. The method of claim 22, wherein the non-oncolytic virus comprises lymphocytic choriomeningitis virus (LCMV).

25. The method of claim 22, wherein the tumour cells comprise B16 tumour cells comprising at least one TAA, at least one TSA, or a combination thereof.

26. The method of claim 22, wherein the tumour cells comprise B16-OVA tumour cells or EL4-OVA tumour cells comprising at least one TAA, at least one TSA, or a combination thereof.
